(19) United States
(12) Patent Application Publication
(10) Pub. No.: US 2026/0202568 A1
(43) Pub. Date: Jul. 16, 2026

(54) FREQUENCY AND AMPLITUDE REDUCTION REGULATION AND CONTROL METHOD AND SYSTEM FOR MULTI-SOURCE DYNAMIC DISTURBANCES IN DEEP TUNNELS

(71) Applicant: NORTHEASTERN UNIVERSITY, Shenyang (CN)

(72) Inventors: Benguo HE, Shenyang (CN); Hengyuan ZHANG, Shenyang (CN); Xiating FENG, Shenyang (CN); Xiangrui MENG, Shenyang (CN); Chong REN, Shenyang (CN); Zihui ZHU, Shenyang (CN)

(21) Appl. No.: 19/331,626

(22) Filed: Sep. 17, 2025

(30) Foreign Application Priority Data
Jan. 15, 2025 (CN) 202510059524.6

(51) Int. Cl. G01V 1/50; G01V 1/28; G01V 1/34; G06N 3/048; G06N 3/08

(52) U.S. Cl. CPC G01V 1/50; G01V 1/282; G01V 1/345; G06N 3/048; G06N 3/08; G01V 2210/32

(57) ABSTRACT

Provided are a frequency and amplitude reduction regulation and control method and system for multi-source dynamic disturbances in deep tunnels. The method includes: acquiring first information and a wave-absorbing material dataset; determining mounting positions of sensors according to the first information, and acquiring second information, wherein the second information includes disturbance wave data collected by the sensors; constructing a first feature diagram according to the second information; determining third information according to the first feature diagram, wherein the third information includes types of disturbance waves; dividing the wave-absorbing material dataset according to the third information to obtain at least one divided wave-absorbing material dataset; and based on the divided wave-absorbing material dataset, training a neural network to obtain at least one trained neural network model, wherein the trained neural network model is configured to output wave-absorbing material mixture ratios corresponding to different types of disturbance waves.

### BACKGROUND OF THE INVENTION

### 1. Field of the Invention

The present invention relates to the field of tunnel excavation construction, and specifically to a frequency and amplitude reduction regulation and control method and system for multi-source dynamic disturbances in deep tunnels.

### 2. The Prior Arts

As tunnel engineering construction progressively extends into deeper geological strata, high-geostress environments induced by deeper depth have become a critical challenge in tunnel construction. Currently, tunnel excavation manners primarily adopt drill-and-blast methods and TBM methods. Regardless of the excavation manners adopted, surrounding rocks are inevitably subjected to dynamic disturbances from blasting waves and TBM vibration waves. Under a superimposed effect of dual adverse factors of high geostress and intense excavation disturbances, repeated multi-source dynamic disturbances induce surrounding rock fracturing and even rockbursts, severely effecting the safe construction of deep tunnels. The current control technologies for failures triggered by dynamic disturbances are relatively scarce. Common ground engineering disturbance control techniques such as isolation trenches (or damping trenches, shock-absorbing trenches) are difficult to implement in deep high-stress tunnels, and lacking of insufficient technologies have led to frequent occurrences of failures triggered by dynamic disturbances on-site.

### SUMMARY OF THE INVENTION

The objective of the present invention is to provide a frequency and amplitude reduction regulation and control method and system for multi-source dynamic disturbances in deep tunnels, thereby solving the above problems.

In order to achieve the objective, the embodiments of the present invention provide the following technical solutions:

In one aspect, the embodiments of the present invention provide the frequency and amplitude reduction regulation and control method for multi-source dynamic disturbances in deep tunnels, the method includes:
Acquiring first information and a wave-absorbing material dataset, where the first information includes measurement point locations of sensors;
Determining mounting positions of the sensors according to the first information, and acquiring second information, where the second information includes disturbance wave data collected by the sensors;
Constructing a first feature diagram according to the second information, where the first feature diagram includes a time-domain diagram and a frequency-domain diagram;
Determining third information according to the first feature diagram, where the third information includes types of disturbance waves;
Dividing the wave-absorbing material dataset according to the third information to obtain at least one divided wave-absorbing material dataset; and
Based on the divided wave-absorbing material dataset, training a neural network to obtain at least one trained neural network model, where the trained neural network model is configured to output wave-absorbing material mixture ratios corresponding to different types of disturbance waves.

In a second aspect, the embodiments of the present invention provide a frequency and amplitude reduction regulation and control system for multi-source dynamic disturbances in deep tunnels, the system includes:
An acquisition module configured to acquire the first information and the wave-absorbing material dataset, wherein the first information includes measurement point locations of sensors;
A first processing module configured to determine mounting positions of the sensors according to the first information, and acquire second information, wherein the second information includes disturbance wave data collected by the sensors;
A second processing module configured to construct a first feature diagram according to the second information, wherein the first feature diagram includes a time-domain diagram and a frequency-domain diagram;
A third processing module configured to determine third information according to the first feature diagram, wherein the third information includes types of disturbance waves;
A fourth processing module configured to divide the wave-absorbing material dataset according to the third information to obtain at least one divided wave-absorbing material dataset; and
A fifth processing module configured to, based on the divided wave-absorbing material dataset, train a neural network to obtain at least one trained neural network model, where the trained neural network model is configured to output wave-absorbing material mixture ratios corresponding to different types of disturbance waves.

In a third aspect, the embodiments of the present invention provide a device, and the device includes a memory and a processor, wherein the memory is configured to store a computer program, and the processor is configured to implement the steps of the frequency and amplitude reduction regulation and control method for multi-source dynamic disturbances in deep tunnels when executing the computer program.

In a fourth aspect, the embodiments of the present invention provide a readable storage medium storing the computer program thereon, when the computer program is executed by the processor, the steps of the frequency and amplitude reduction regulation and control method for multi-source dynamic disturbances in deep tunnels are implemented.

The present invention has the beneficial effects:

The mounting positions of the sensors are determined by means of the first information, the disturbance wave data is collected by using the sensors to construct the first feature diagram, the types of the disturbance waves are determined according to the first feature diagram, the wave-absorbing material dataset is divided according to the types of the disturbance waves, and by using the divided wave-absorbing material dataset, the neural network is trained to obtain at least one trained neural network model. According to the present invention, based on the types of the disturbance waves, the wave-absorbing material dataset is divided, thereby improving the quality of the dataset, improving the accuracy of outputted wave-absorbing material mixture ratios and achieving the purpose of improving frequency and amplitude reduction effects.

Additional features and advantages of the present invention will be set forth in the subsequent description, some will become apparent therefrom, or may be understood through implementation of the embodiments. The objectives and other advantages of the present invention may be realized and obtained through the structures particularly pointed out in the written description and claims hereof, and the appended drawings.

### BRIEF DESCRIPTION OF DRAWINGS

In order to describe the technical solutions of the embodiments of the present invention more clearly, the accompanying drawings required for the embodiments will be briefly introduced below. It should be understood that these drawings illustrate only certain embodiments of the present invention and therefore shall not be construed as limiting the scope. For a person of ordinary skill in the art, other related drawings may be obtained according to these illustrations without creative effort.

FIG. 1 is a schematic flow diagram of a frequency and amplitude reduction regulation and control method for multi-source dynamic disturbances in deep tunnels according to an embodiment of the present invention.

FIG. 2 is a schematic structural diagram of a frequency and amplitude reduction regulation and control system for multi-source dynamic disturbances in deep tunnels according to an embodiment of the present invention.

FIG. 3 is a schematic structural diagram of a frequency and amplitude reduction regulation and control device for multi-source dynamic disturbances in deep tunnels according to an embodiment of the present invention.

FIG. 4 is a fourth feature diagram according to an embodiment of the present invention.

FIG. 5 is a diagram of a relationship between fiber ratios and an attenuation ratio of peak vibration velocity according to an embodiment of the present invention.

### DETAILED DESCRIPTION OF THE PREFERRED EMBODIMENT

In order to make the objectives, technical solutions, and advantages of the embodiments of the present invention clearer, the following clearly and completely describes the technical solutions in the embodiments of the present invention with reference to the accompanying drawings in the embodiments of the present invention. Apparently, the described embodiments are merely a part rather than all of the embodiments of the present invention. Usually, components of the embodiments of the present invention described and illustrated in the accompanying drawings herein may be arranged and designed in a variety of configurations. Therefore, the following detailed description of the embodiments of the present invention provided in the accompanying drawings is not intended to limit the scope of the present invention which is required to be protected, but merely intended to represent selected embodiments of the present invention. On the basis of the embodiments of the present invention, all other embodiments acquired by those skilled in the art without creative effort fall within the scope of protection of the present invention.

It should be noted that: similar reference characters and numerals in the following drawings denote similar items. Therefore, once an item is defined in one drawing, it may not be further defined or explained in subsequent drawings. Moreover, in the description of the present invention, the terms such as “first” and “second” are only for distinguishing the description, not for indicating or implying the relative importance.

### Embodiment 1

The present embodiment provides a frequency and amplitude reduction regulation and control method for multi-source dynamic disturbances in deep tunnels. It should be understood that an application scenario may be established herein, for example: during blasting excavation of a deep tunnel, a scenario where disturbance waves induce fracturing of surrounding rocks, and thus it is difficult to continue construction exists.

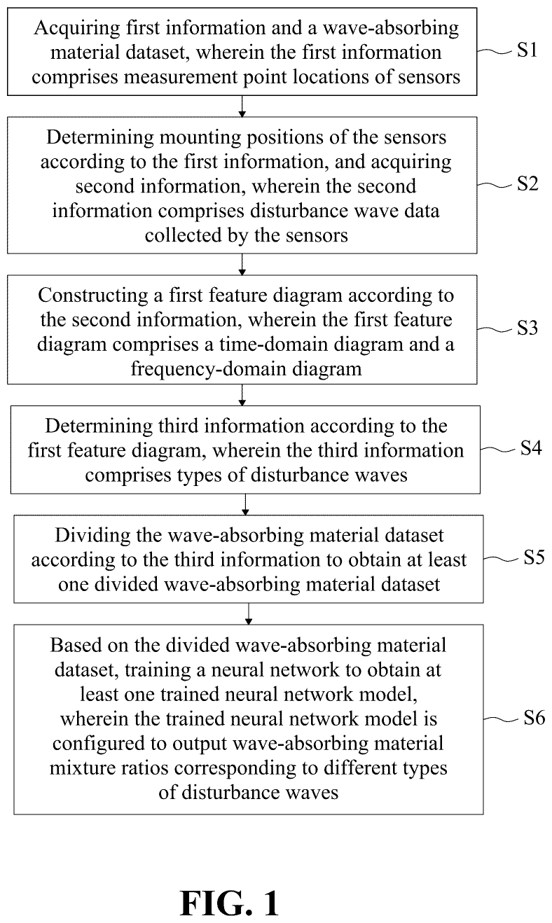

Referring to FIG. 1, which shows that the method includes Steps S1, S2, S3, S4, S5, and S6.

In Step S1, first information and a wave-absorbing material dataset are acquired, wherein the first information includes measurement point locations of sensors; and

In the present step, the wave-absorbing material dataset includes mixture ratios of different wave-absorbing materials, including but not limited to: cement mortar, foaming agents, basalt fibers, ceramic fibers, gypsum, rubber powder, and polyacrylonitrile fibers.

Step S1 further includes Steps S11, S12, S13 and S14, specifically including:
In Step S11: microseismic monitoring warning data is acquired;
In Step S12: a fourth feature diagram is constructed according to the microseismic monitoring warning data, wherein the fourth feature diagram includes a relationship between a distance from an excavation site to a tunnel face and a microseismic event occurrence ratio; and

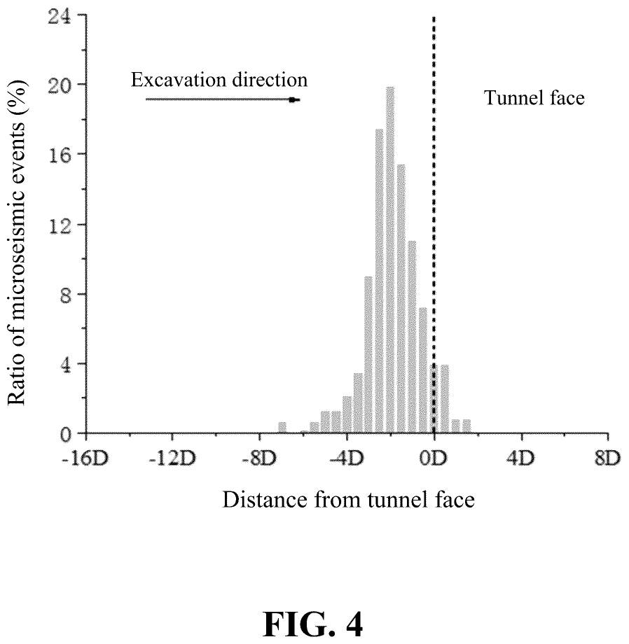

In the present step, the fourth feature diagram is specifically shown as FIG. 4, when a distance from the excavation site to the tunnel face is relatively short, massive microseismic events occur, leading to a high microseismic event occurrence ratio. Consequently, a grouting zone for wave-absorbing materials should not be deployed too close to the tunnel face, otherwise, the grouting zone of the wave-absorbing materials will be damaged, and wave-absorbing efficacy will be influenced.

In Step S13, the grouting zone of the wave-absorbing materials is determined according to the fourth feature diagram; and

In the present step, a specific implementation lies in that: massive microseismic events are prone to occur within a range of 2-3 times a tunnel diameter from the excavation site to the tunnel face, and stress adjustment mainly occurs within the range; and therefore, wave-absorbing grouting zones are arranged at 3 times the tunnel diameter from the excavation site, thereby ensuring the grouting zone of the wave-absorbing materials not to be destroyed, avoiding the disturbance waves from natural attenuation with increasing distances.

In Step S14, the measurement point locations of the sensors are determined according to the grouting zone of the wave-absorbing materials.

In Step S2, mounting positions of the sensors are determined according to the first information, and second information is acquired, wherein the second information includes disturbance wave data collected by the sensors; and

In the present step, after the grouting zone of the wave-absorbing materials is determined, two monitoring holes of 10 cm in diameter and 10 cm in depth are provided in positions 10 cm in front of and behind a grouting hole, and the sensors are placed at the monitoring holes, and fixed by using high-strength fast-setting gypsum.

In Step S3, a first feature diagram is constructed according to the second information, wherein the first feature diagram includes a time-domain diagram and a frequency-domain diagram;

In Step S4, third information is determined according to the first feature diagram, wherein the third information includes types of the disturbance waves;

The Step S4 further includes Steps S41, S42 and S43, specifically including:

In Step S41, a vibration frequency of the disturbance waves is determined according to the frequency-domain diagram;

In Step S42, an amplitude of the disturbance waves at each time point is extracted from the time-domain diagram to obtain an amplitude distribution range of the disturbance waves; and

In Step S43, the types of the disturbance waves are determined according to the vibration frequency of the disturbance waves and the amplitude distribution range of the disturbance waves.

In the present embodiment, Fourier transform is performed on waveforms of the disturbance waves through MATLAB, obtaining the following frequency and amplitude distribution ranges for different types of disturbance waves: seismic waves have a frequency of 5-20 Hz, and an amplitude of 10-100 MPa; blasting waves of the tunnel face have a frequency of 100-500 Hz, and an amplitude of 10-100 MPa; TBM mechanical vibration waves have a frequency of 800-1200 Hz, and an amplitude of 1-10 MPa; and rockburst stress waves have a frequency of 100-500 Hz, and an amplitude of 10-200 MPa, such that the types of the disturbance waves can be rapidly identified according to corresponding time-domain diagrams.

In Step S5, the wave-absorbing material dataset is divided according to the third information to obtain at least one divided wave-absorbing material dataset; and

In the present step, different wave-absorbing materials exhibit different frequency and amplitude reduction effects on different types of disturbance waves, the wave-absorbing material dataset needs to be divided, thereby improving the quality of the wave-absorbing material dataset.

Step S5 further includes Steps S51, S52, S53 and S54, specifically including:

In Step S51, pre-processing is performed according to the time-domain diagram corresponding to the third information to obtain a pre-processed waveform diagram.

In the present step, the pre-processing mainly includes: removing abnormal values, performing waveform noise reduction processing, and averaging waveform parameters.

In Step S52, the disturbance wave feature parameters are extracted from the pre-processed waveform diagram; and

In the present step, the disturbance wave feature parameters include but are not limited to peak vibration velocity and frequency.

In Step S53, the attenuation effect of each wave-absorbing material in the wave-absorbing material dataset on the disturbance wave feature parameters is determined to obtain fourth information; and

In the present step, wave-absorbing materials showing the most significant amplitude attenuation effects need to be selected for regulating and controlling high-amplitude disturbance waves, while wave-absorbing materials showing the most frequency attenuation effects need to be selected for regulating and controlling high-frequency disturbance waves. Consequently, the wave-absorbing material dataset for different types of disturbance waves can be screened through the attenuation effects of each wave-absorbing material on the disturbance wave feature parameters.

Step S53 further includes Steps S531, S532, S533 and S534, specifically including:
In Step S531, a second feature diagram and a third feature diagram are acquired, wherein the second feature diagram includes a waveform diagram of the disturbance waves before the wave-absorbing materials are ungrouted, and the third feature diagram includes a waveform diagram of the disturbance waves after the wave-absorbing materials are grouted;
In Step S532, a difference value between peak vibration velocities at each time point in the second feature diagram and the third feature diagram is calculated to obtain a first calculation result;
In Step S533, the first calculation result at each time point is compared with a corresponding vibration velocity at each time point in the second feature diagram to obtain ratio results; and
In the present step, the ratio results represent an amplitude attenuation ratio of the disturbance waves at each time point before and after wave absorption.

In Step S534, an average value of all of the ratio results is calculated to obtain an amplitude attenuation effect of the disturbance waves.

In the present step, the amplitude attenuation effect of the wave-absorbing materials on the disturbance waves can be quantified by calculating the average value of the amplitude attenuation ratio at each time point, thereby providing a data basis for subsequent dataset division. It should be noted that the attenuation effects of the disturbance waves on the frequency can also be determined by calculating the difference value in frequency attenuation of the disturbance waves at each time point.

In Step S54, the wave-absorbing material dataset is divided according to the fourth information and the third information.

In Step S6, based on the divided wave-absorbing material dataset, a neural network is trained to obtain at least one trained neural network model, wherein the trained neural network model is configured to output wave-absorbing material mixture ratios corresponding to different types of disturbance waves.

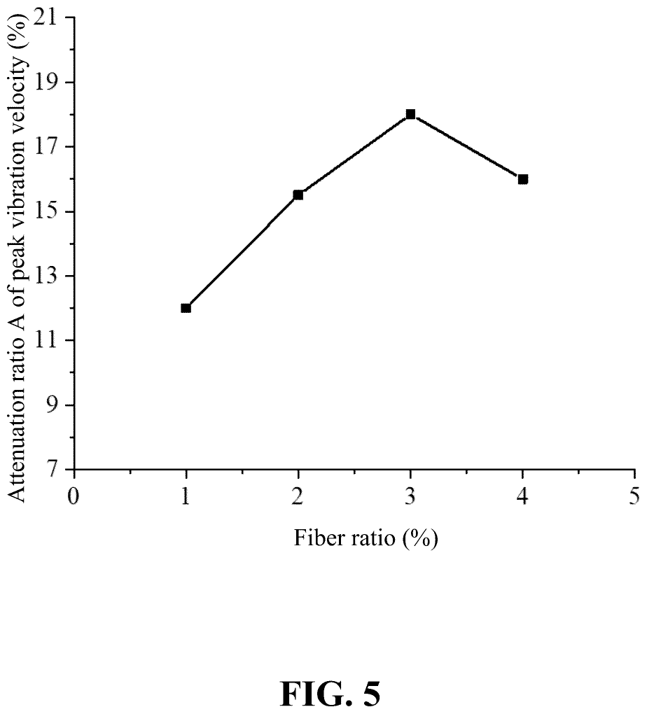

Based on the divided wave-absorbing material dataset, a sample set is constructed. The sample set includes a mapping relationship between different types of materials and the disturbance wave feature parameters, as shown in FIG. 5, representing a relationship between a fiber ratio and an attenuation ratio of peak vibration velocity. Besides, in the present step, since the divided wave-absorbing material dataset is obtained after division on the basis of wave-absorption types, wherein a large quantity of materials exhibiting a poor regulation and control effect on this type of disturbance waves have been filtered out, only a mapping relationship between the materials exhibiting a good regulation and control effect on this type of disturbance waves in the divided wave-absorbing material dataset and the disturbance wave feature parameters needs to be established, thereby greatly increasing a construction speed of the sample set to shorten training time of a model. Furthermore, multiple sample sets can be constructed through the divided wave-absorbing material dataset, each sample set adapts to one type of disturbance waves, thereby obtaining a plurality of trained neural network models, improving the precision of a wave-absorbing material ratio, and achieving the purpose of enhancing frequency and amplitude reduction effects of the disturbance waves.

Step S6 further includes Steps S61, S62, S63 and S64, specifically including:
In Step S61, connection weight values between a hidden layer and an output layer of the neural network are calculated by using a least squares method;
In Step S62, a predefined activation function of the neural network is determined according to the connection weight values;
In Step S63, the disturbance wave feature parameters are acquired according to the time-domain diagram and the frequency-domain diagram; and
In Step S64, normalization processing is performed on the disturbance wave feature parameters to obtain input samples for an activation function of the neural network.

In the present embodiment, an RBF neural network is used to construct a deep learning artificial network identification model. The RBF neural network is a three-layer neural network including an input layer, a hidden layer, and an output layer. Transformation from the input layer to the hidden layer is nonlinear, while transformation from the hidden layer to the output layer is linear, wherein a predefined activation function is specifically defined as follows:

$$R ( {x}^{P} - {c}^{p} ) ≡ exp ( - \frac{1}{2 {σ}^{2}} {‖ {x}^{p} - {c}^{p} ‖}^{2} )$$

In the above formula: R represents the predefined activation function of the RBF neural network,

$${x}^{p} = [ {x}_{1}^{p} , {x}_{2}^{p} , … , {x}_{m}^{p} ]$$

represents a p^{th} input sample, wherein the sample content includes vibration velocity and frequency of the disturbance waves;

$${c}^{p} = [ {c}_{1}^{p} , {x}_{2}^{p} , … , {c}_{m}^{p} ]$$

represents a center vector of a radial basis function for a p^{th} hidden layer node, m is the total number of samples, and ∥x^{p}−c^{p}∥ is a Euclidean norm; σ is a variance of a Gaussian function, and determined by solving weight values between the hidden layer and the output layer, namely

$${σ}_{i} = \frac{{c}_{max}}{\sqrt{2 h}} ;$$

i=1, 2, . . . , h, wherein cₘₐₓ is a maximum distance between selected cluster center points, and h is the total number of cluster centers.

The RBF neural network is trained through the sample set until grid convergence is achieved, a set error criterion is met, a trained deep learning artificial network identification model, namely a trained neural network model, is obtained, the monitored disturbance wave feature parameters (peak vibration velocity and frequency) are uploaded to a terminal device, and the trained neural network model can output an optimal wave-absorbing material and a ratio, thereby achieving frequency and amplitude reduction regulation and control on the disturbance waves.

### Embodiment 2

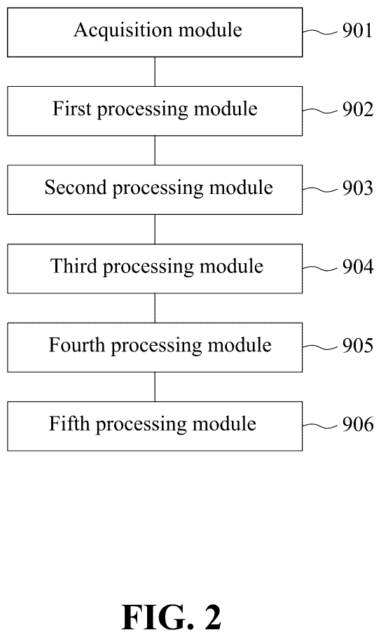

As shown in FIG. 2, the present embodiment provides a frequency and amplitude reduction regulation and control system for multi-source dynamic disturbances in deep tunnels. The system includes an acquisition module 901, a first processing module 902, a second processing module 903, a third processing module 904, a fourth processing module 905 and a fifth processing module 906, specifically including:
The acquisition module 901 configured to acquire first information and a wave-absorbing material dataset, wherein the first information includes the measurement point locations of the sensors;
The first processing module 902 configured to determine mounting positions of the sensors according to the first information, and acquire second information, wherein the second information includes disturbance wave data collected by the sensors;
The second processing module 903 configured to construct a first feature diagram according to the second information, wherein the first feature diagram includes a time-domain diagram and a frequency-domain diagram;
The third processing module 904 configured to determine third information according to the first feature diagram, wherein the third information includes types of disturbance waves;
The fourth processing module 905 configured to divide the wave-absorbing material dataset according to the third information to obtain at least one divided wave-absorbing material dataset; and
The fifth processing module 906 configured to, based on the divided wave-absorbing material dataset, train a neural network to obtain at least one trained neural network model, wherein the trained neural network model is configured to output wave-absorbing material mixture ratios corresponding to different types of disturbance waves.

In one specific implementation of the present invention, the third processing module further includes a first processing unit, a second processing unit and a third processing unit, specifically including:
The first processing unit configured to determine a vibration frequency of the disturbance waves according to the frequency-domain diagram;
The second processing unit configured to extract an amplitude of the disturbance waves at each time point from the time-domain diagram to obtain an amplitude distribution range of the disturbance waves; and
The third processing unit configured to determine the types of the disturbance waves according to the vibration frequency of the disturbance waves and the amplitude distribution range of the disturbance waves.

In one specific implementation of the present invention, the fourth processing module further includes a pre-processing unit, a fourth processing unit, a fifth processing unit and a sixth processing unit, specifically including:
The pre-processing unit configured to perform pre-processing according to the time-domain diagram corresponding to the third information to obtain a pre-processed waveform diagram;
The fourth processing unit configured to extract disturbance wave feature parameters from the pre-processed waveform diagram;
The fifth processing unit configured to determine an attenuation effect of each wave-absorbing material in the wave-absorbing material dataset on the disturbance wave feature parameters to obtain fourth information; and
The sixth processing unit configured to divide the wave-absorbing material dataset according to the fourth information and the third information.

In one specific implementation of the present invention, the fifth processing module further includes a first acquisition unit, a seventh processing unit, an eighth processing unit and a ninth processing unit, specifically including:
The first acquisition unit configured to acquire a second feature diagram and a third feature diagram, wherein the second feature diagram includes a waveform diagram of the disturbance waves before the wave-absorbing materials are ungrouted, and the third feature diagram includes a waveform diagram of the disturbance waves after the wave-absorbing materials are grouted;
The seventh processing unit configured to calculate a difference value between vibration velocities at each time point in the second feature diagram and the third feature diagram to obtain a first calculation result;
The eighth processing unit configured to compare the first calculation result at each time point with a corresponding vibration velocity at each time point in the second feature diagram to obtain ratio results; and
The ninth processing unit configured to calculate an average value of all of the ratio results to obtain an amplitude attenuation effect of the disturbance waves.

In one specific implementation of the present invention, the fifth processing module further includes a tenth processing unit, an eleventh processing unit, a twelfth processing unit and a thirteenth processing unit, specifically including:
The tenth processing unit configured to calculate connection weight values between a hidden layer and an output layer of a neural network by using a least squares method;
The eleventh processing unit configured to determine a predefined activation function of the neural network according to the connection weight values;
The twelfth processing unit configured to acquire the disturbance wave feature parameters according to the time-domain diagram and the frequency-domain diagram; and
The thirteenth processing unit configured to perform normalization processing on the disturbance wave feature parameters to obtain input samples for an activation function of the neural network.

In one specific implementation of the present invention, the acquisition module further includes a second acquisition unit, a fourteenth processing unit, a fifteenth processing unit and a sixteenth processing unit, specifically including:
The second acquisition unit configured to acquire microseismic monitoring warning data;
The fourteenth processing unit configured to construct a fourth feature diagram according to the microseismic monitoring warning data, wherein the fourth feature diagram includes a relationship between a distance from an excavation site to a tunnel face and a microseismic event occurrence ratio;
The fifteenth processing unit configured to determine a grouting zone of the wave-absorbing materials according to the fourth feature diagram; and
The sixteenth processing unit configured to determine measurement point locations of the sensors according to the grouting zone of the wave-absorbing materials.

It should be noted that regarding the system described in the above embodiment, the implementation performed by each module has been described in detail in the relevant method embodiments. Therefore, a detailed description will not be repeated here.

### Embodiment 3

Corresponding to the above method embodiment, the present embodiment further provides a frequency and amplitude reduction regulation and control device for multi-source dynamic disturbances in deep tunnels. The frequency and amplitude reduction regulation and control device for multi-source dynamic disturbances in deep tunnels described below and the frequency and amplitude reduction regulation and control method for multi-source dynamic disturbances in deep tunnels described above may be cross-referenced.

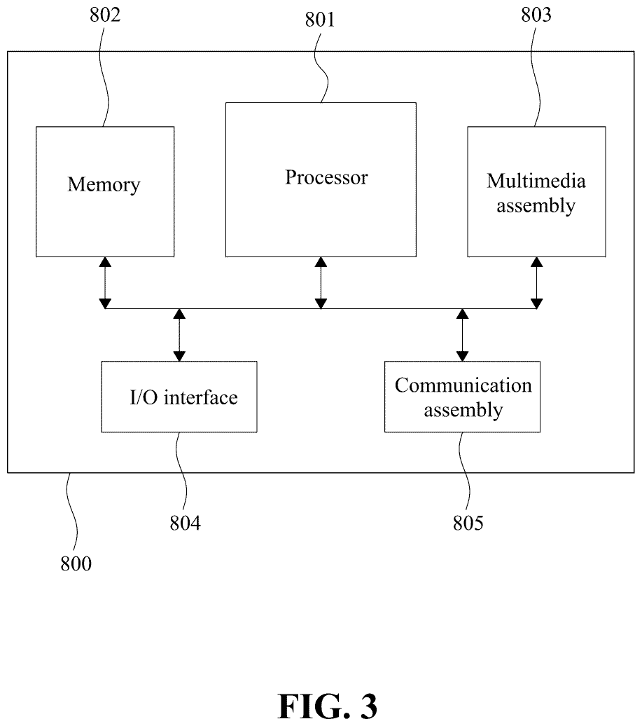

FIG. 3 is a block diagram of the frequency and amplitude reduction regulation and control device 800 for multi-source dynamic disturbances in deep tunnels according to an exemplary embodiment. As shown in FIG. 3, the frequency and amplitude reduction regulation and control device 800 for multi-source dynamic disturbances in deep tunnels includes a processor 801 and a memory 802. The frequency and amplitude reduction regulation and control device 800 for multi-source dynamic disturbances in deep tunnels can further include one or more of a multimedia assembly 803, an I/O interface 804 and a communication assembly 805.

The processor 801 is configured to control the overall operation of the frequency and amplitude reduction regulation and control device 800 for multi-source dynamic disturbances in deep tunnels, so as to complete all or part of the steps in the frequency and amplitude reduction regulation and control method for multi-source dynamic disturbances in deep tunnels. The memory 802 is configured to store various types of data to support the operation of the frequency and amplitude reduction regulation and control device 800 for multi-source dynamic disturbances in deep tunnels. Such data may include, for example, instructions for any applications or methods operated on the frequency and amplitude reduction regulation and control device 800 for multi-source dynamic disturbances in deep tunnels, as well as application-related data, such as contact data, sent and received messages, pictures, audio and video. The memory 802 may be implemented by any type of volatile or non-volatile storage devices or a combination thereof, for example, static random access memory (SRAM), electrically erasable programmable read-only memory (EEPROM), erasable programmable read-only memory (EPROM), programmable read-only memory (PROM), read-only memory (ROM), magnetic memory, flash memory, disks or optical disks. The multimedia assembly 803 may include a screen and an audio assembly, wherein the screen may be, for example, a touch screen, and the audio assembly is configured to output and/or input audio signals. For example, the audio assembly may include a microphone which is configured to receive external audio signals. The received audio signals may be further stored in the memory 802 or sent via the communication assembly 805. The audio assembly further includes at least one speaker configured to output the audio signals. The I/O interface 804 provides an interface between the processor 801 and other interface modules. The other interface modules may be a keyboard, a mouse, buttons, etc. These buttons may be virtualized buttons or mechanical buttons. The communication assembly 805 is configured to enable wired or wireless communication between the frequency and amplitude reduction regulation and control device 800 for multi-source dynamic disturbances in deep tunnels and other devices. Wireless communication refers to, for example, Wi-Fi, Bluetooth, near field communication (NFC), 2G, 3G, or 4G, or a combination of one or more thereof, therefore the corresponding communication assembly 805 may include: a Wi-Fi module, a Bluetooth module and an NFC module.

In an exemplary embodiment, the frequency and amplitude reduction regulation and control device 800 for multi-source dynamic disturbances in deep tunnels may be implemented by one or more of the following: an application specific integrated circuit (ASIC), a digital signal processor (DSP), a digital signal processing device (DSPD), a programmable logic device (PLD), a field programmable gate array (FPGA), a controller, a microcontroller, a microprocessor, or other electronic components, which are configured to execute the frequency and amplitude reduction regulation and control method for multi-source dynamic disturbances in deep tunnels.

In another exemplary embodiment, further provided is a computer-readable storage medium containing program instructions. When the program instructions are executed by the processor, the steps of the frequency and amplitude reduction regulation and control method for multi-source dynamic disturbances in deep tunnels are implemented. For example, the computer-readable storage medium may be the above memory 802 containing the program instructions. The above program instructions may be executed by the processor 801 of the frequency and amplitude reduction regulation and control device 800 for multi-source dynamic disturbances in deep tunnels so as to complete the frequency and amplitude reduction regulation and control method for multi-source dynamic disturbances in deep tunnels.

### Embodiment 4

Corresponding to the above method embodiment, the present embodiment further provides a readable storage medium. The readable storage medium described below and a frequency and amplitude reduction regulation and control method for multi-source dynamic disturbances in deep tunnels described above may be cross-referenced.

The readable storage medium stores a computer program thereon, when the computer program is executed by the processor, the steps of the frequency and amplitude reduction regulation and control method for multi-source dynamic disturbances in deep tunnels in the method of the embodiments are implemented.

The readable storage medium may specifically be any readable storage medium capable of storing program codes, such as a USB disk, a mobile hard disk, a read-only memory (ROM), a random access memory (RAM), a magnetic disk, or an optical disk.

The foregoing are merely preferred embodiments of the present invention and are not intended to limit the scope. For those skilled in the art, various modifications and changes may be made to the present invention. Any amendments, equivalent substitutions, improvements, etc., made within the spirit and principles of the present invention shall fall within the scope of protection of the present invention.

The above descriptions are merely specific implementations of the present invention, any person skilled in the art could readily conceive of variations or substitutions within the technical scope disclosed by the present invention, and these improvements and substitutions should be encompassed within the protection scope of the present invention. Therefore, the protection scope of the present invention shall be defined by the claims.

What is claimed is:

1. A frequency and amplitude reduction regulation and control method for multi-source dynamic disturbances in deep tunnels, comprising:
acquiring first information and a wave-absorbing material dataset, wherein the first information comprises measurement point locations of sensors;
determining mounting positions of the sensors according to the first information, and acquiring second information, wherein the second information comprises disturbance wave data collected by the sensors;
constructing a first feature diagram according to the second information, wherein the first feature diagram comprises a time-domain diagram and a frequency-domain diagram;
determining third information according to the first feature diagram, wherein the third information comprises types of disturbance waves;
dividing the wave-absorbing material dataset according to the third information to obtain at least one divided wave-absorbing material dataset; and
based on the divided wave-absorbing material dataset, training a neural network to obtain at least one trained neural network model, wherein the trained neural network model is configured to output wave-absorbing material mixture ratios corresponding to different types of disturbance waves,
wherein the dividing the wave-absorbing material dataset according to the third information comprises:
performing pre-processing according to the time-domain diagram corresponding to the third information to obtain a pre-processed waveform diagram;
extracting disturbance wave feature parameters from the pre-processed waveform diagram;
determining an attenuation effect of each wave-absorbing material in the wave-absorbing material dataset on the disturbance wave feature parameters to obtain fourth information;
dividing the wave-absorbing material dataset according to the fourth information and the third information,
wherein the determining the attenuation effect of each wave-absorbing material in the wave-absorbing material dataset on the disturbance wave feature parameters comprises:
acquiring a second feature diagram and a third feature diagram, wherein the second feature diagram comprises a waveform diagram of the disturbance waves before the wave-absorbing materials are ungrouted, and the third feature diagram comprises a waveform diagram of the disturbance waves after the wave-absorbing materials are grouted;
calculating a difference value between vibration velocities at each time point in the second feature diagram and the third feature diagram to obtain a first calculation result;
comparing the first calculation result at each time point with a corresponding vibration velocity at each time point in the second feature diagram to obtain ratio results; and
calculating an average value of all of the ratio results to obtain an amplitude attenuation effect of the disturbance waves.

2. The frequency and amplitude reduction regulation and control method according to claim 1, wherein the determining third information according to the first feature diagram, wherein the third information comprises types of disturbance waves, comprises:
determining a vibration frequency of the disturbance waves according to the frequency-domain diagram;
extracting an amplitude of the disturbance waves at each time point from the time-domain diagram to obtain an amplitude distribution range of the disturbance waves; and
determining the types of the disturbance waves according to the vibration frequency of the disturbance waves and the amplitude distribution range of the disturbance waves.

3. The frequency and amplitude reduction regulation and control method according to claim 1, wherein based on the divided wave-absorbing material dataset, training the neural network to obtain the at least one trained neural network model, comprises:
calculating connection weight values between a hidden layer and an output layer of the neural network by using a least squares method;
determining a predefined activation function of the neural network according to the connection weight values;
acquiring the disturbance wave feature parameters according to the time-domain diagram and the frequency-domain diagram; and
performing normalization processing on the disturbance wave feature parameters to obtain input samples for an activation function of the neural network.

4. A frequency and amplitude reduction regulation and control system for multi-source dynamic disturbances in deep tunnels, comprising:
an acquisition module configured to acquire first information and a wave-absorbing material dataset, wherein the first information comprises measurement point locations of sensors;
a first processing module configured to determine mounting positions of the sensors according to the first information, and acquire second information, wherein the second information comprises disturbance wave data collected by the sensors;
a second processing module configured to construct a first feature diagram according to the second information, wherein the first feature diagram comprises a time-domain diagram and a frequency-domain diagram;
a third processing module configured to determine third information according to the first feature diagram, wherein the third information comprises types of disturbance waves;
a fourth processing module configured to divide the wave-absorbing material dataset according to the third information to obtain at least one divided wave-absorbing material dataset; and
a fifth processing module configured to, based on the divided wave-absorbing material dataset, train a neural network to obtain at least one trained neural network model, wherein the trained neural network model is configured to output wave-absorbing material mixture ratios corresponding to different types of disturbance waves,
wherein the fourth processing module comprises:
a pre-processing unit configured to perform pre-processing according to the time-domain diagram corresponding to the third information to obtain a pre-processed waveform diagram;
a fourth processing unit configured to extract disturbance wave feature parameters from the pre-processed waveform diagram;
a fifth processing unit configured to determine an attenuation effect of each wave-absorbing material in the wave-absorbing material dataset on the disturbance wave feature parameters to obtain fourth information; and
a sixth processing unit configured to divide the wave-absorbing material dataset according to the fourth information and the third information,
wherein the fifth processing unit comprises:
a first acquisition unit configured to acquire a second feature diagram and a third feature diagram, wherein the second feature diagram comprises a waveform diagram of the disturbance waves before the wave-absorbing materials are ungrouted, and the third feature diagram comprises a waveform diagram of the disturbance waves after the wave-absorbing materials are grouted;
a seventh processing unit configured to calculate a difference value between vibration velocities at each time point in the second feature diagram and the third feature diagram to obtain a first calculation result;
an eighth processing unit configured to compare the first calculation result at each time point with a corresponding vibration velocity at each time point in the second feature diagram to obtain ratio results; and
a ninth processing unit configured to calculate an average value of all of the ratio results to obtain an amplitude attenuation effect of the disturbance waves.

5. The frequency and amplitude reduction regulation and control system according to claim 4, wherein the third processing module comprises:
a first processing unit configured to determine a vibration frequency of the disturbance waves according to the frequency-domain diagram;
a second processing unit configured to extract an amplitude of the disturbance waves at each time point from the time-domain diagram to obtain an amplitude distribution range of the disturbance waves; and
a third processing unit configured to determine the types of the disturbance waves according to the vibration frequency of the disturbance waves and the amplitude distribution range of the disturbance waves.

6. The frequency and amplitude reduction regulation and control system according to claim 4, wherein the fifth processing module comprises:
a tenth processing unit configured to calculate connection weight values between a hidden layer and an output layer of a neural network by using a least squares method;
an eleventh processing unit configured to determine a predefined activation function of the neural network according to the connection weight values;
a twelfth processing unit configured to acquire the disturbance wave feature parameters according to the time-domain diagram and the frequency-domain diagram; and
a thirteenth processing unit configured to perform normalization processing on the disturbance wave feature parameters to obtain input samples for an activation function of the neural network.
